(19) United States
(12) Patent Application Publication
(10) Pub. No.: US 2013/0072763 A1
(43) Pub. Date: Mar. 21, 2013

(54) Mobile Monitoring Device for Monitoring Physical Characteristics of a Subject

(71) Applicant: Haim Shtalryd, Ness Ziyona (IL)

(72) Inventor: Haim Shtalryd, Ness Ziyona (IL)

(21) Appl. No.: 13/616,443

(22) Filed: Sep. 14, 2012

Related U.S. Application Data
(60) Provisional application No. 61/534,992, filed on Sep. 15, 2011.

(51) Int. Cl. A61B 5/08; A61B 5/01; A61B 5/113

(52) U.S. Cl. 600/301; 600/534

(57) ABSTRACT

A mobile monitoring device (MMD) for monitoring physical characteristics of a subject, including a casing; an attaching mechanism for releasably attaching the MMD to a garment worn by the subject; a control unit enabling to control and execute operations of the MMD and identify breathing related alarming situations; a sensor element enabling to sense breathing related characteristics of the subject by converting physical vibrations into electric signals; an alarm output device for enabling to operate at least one alarm in case alarm breathing related situation is identified; and an arm extending from the casing. The arm allows abutting the body of the subject at a first proximal end thereof and the sensor element at a second distal end thereof when the device is worn by the subject, thereby allows mechanically transmitting physical movements thereof caused by movements of the subject body to the sensor element thereby mechanically leveraging the movement.

### CROSS-REFERENCE TO RELATED PATENT APPLICATIONS

This application claims priority to provisional patent application No. 61/534,992 filed on Sep. 15, 2011, which is incorporated herein by reference in its entirety.

### FIELD OF THE INVENTION

The present invention generally relates to devices, apparatuses, systems and/or methods for monitoring physical characteristics of subjects.

### BACKGROUND OF THE INVENTION

Several types of monitoring systems and devices for monitoring breathing activity of human subjects and especially for apnea detection of babies are currently available. These systems monitor a breathing pattern of the subject and operate an alarm mechanism in case breathing irregularity is detected.

Some systems for monitoring baby breathing include one or more sensor-platforms for placing underneath the baby's mattress to monitor his/her breathing while the baby is sleeping, and one or more control devices that usually hang over the subject's bed. These platforms are quite large in scale typically having surface dimensions of about a quarter of the surface of the entire mattress. Each control device includes the electronic circuitry for operating the system and indication and alarm elements for indicating that the system is operated, battery condition and typically sound alarm elements (e.g. a speaker) for outputting a sound alarm once breathing irregularity is detected.

These systems can only be used if the sensor-platform(s) is (are) placed under the sleeping mattress of the subject (baby) and cannot be used in case the subject is awake or is out of bed. It can also be quite uncomfortable to move such a system from one bed or any other sleeping furniture to another in case, for example, a parent wishes to take his/her baby on camping and still requires monitoring the baby's breathing by moving the entire system from the baby's bed to a camping bed.

A patent application No. US 2010/0201524 A1 (Ser. No. 12/526,590) by Gregory John Gallagher, discloses a method and device for monitoring regular movement of a human body, such as an infant's body. The method includes attaching the device to the body with a protuberance of the body in abutment with the body, so that the protuberance is deflected as the body moves regularly. The deflection of the protuberance is monitored, using a transducer attached to the protuberance and an alarm is activated if the pattern of deflection of the protuberance changes to a predetermined extent, e.g. if it is disrupted. The device includes a clip to be able to attach to the baby's diaper or trousers.

A patent No. US 2002/0124295 A1 (Ser. No. 10/000,181) discloses a clothing apparatus with at least one fabric panel, a panel of stretch fabric, a sensor unit retaining pocket, and a light opaque fabric. The fabric panel is configured to encompass a patient thoracic region. The stretch fabric panel is joined to the fabric panel about the thoracic region of a patient when wearing the clothing apparatus. A free edge of the overlapping stretch fabric panel is configured to be releasably mated along an overlapping area of the stretch fabric panel with one of the fabric panel and the stretch fabric panel. The sensor unit retaining pocket is provided between the at least one fabric panel and the stretch fabric panel. The light opaque fabric is provided in the fabric panel about an opening in an inner surface of the fabric panel contiguous with the retaining pocket. The opening is configured to enable a sensor unit received within the pocket to maintain direct contact with a skin surface of a patient over the thoracic heart region. The light opaque fabric is configured to reduce ambient light levels immediately surrounding the pocket opening.

### SUMMARY OF THE INVENTION

According to some embodiments of the present invention, there is provided a mobile monitoring device for monitoring physical characteristics of a subject. The mobile monitoring device comprises a casing; an attaching mechanism, connected to the casing, for releasably attaching the mobile monitoring device to a garment worn by the subject; a control unit enabling to control and execute operations of the mobile monitoring device and identify breathing related alarming situations; at least one sensor element enabling to sense breathing related characteristics of the subject by converting physical vibrations into electric signals, the sensor element is operatively connected to the control unit; an alarm output device for enabling to operate at least one alarm in case alarm breathing related situation is identified; and at least one arm extending from the casing. The arm allows abutting a body portion of the subject at a first proximal end thereof and the sensor element at a second distal end thereof when the device is worn by the subject, wherein the sensor element is located within the casing. The arm mechanically connects to the sensor element to allow mechanically transmitting physical movements of the arm caused by movements of the subject body to the sensor element thereby mechanically leveraging the movement.

According to some embodiments, the arm comprises a first arm portion and a second arm portion connected thereto. The first arm portion is made from a flexible material, wherein the first arm portion is position angularly to the second arm portion to allow comfortable abutting of the subject's body while wearing the mobile monitoring device.

Optionally, the arm further comprises a third arm portion pivotally connected to the second arm portion. The third arm portion pivotally connects to the second arm portion via a hinge or is integrally connected thereto and is made from a flexible material allowing movement of the third arm portion in relation to the second arm portion thereby.

According to some embodiments, the third arm portion is longer than the overall length of the first and second arm portions combined.

Optionally, a coiled spring connects to the third arm portion at one end and to the second arm portion at another end.

Additionally or alternatively, the sensor element comprises a piezoelectric transducer enabling to convert mechanical force applied upon a surface thereof by the second distal end of the arm into an electric signal.

According to some embodiments, the attaching mechanism comprises a clip for releasably attaching the mobile monitoring device to the garment.

According to some embodiments, the mobile monitoring device further comprises at least one speaker for outputting sound alarms, where the control unit allows identification of a respective breathing related alarming situation by analyzing movement pattern of the arm and operating the sound alarm once identifying the breathing related alarming situation.

The mobile monitoring optionally further comprises a temperature monitoring mechanism. The temperature monitoring mechanism comprises at least one temperature sensor operatively connected to the control unit enabling measuring temperature of an area of the subject's body, wherein the mobile monitoring device enables presenting measured temperature over a designated display unit embedded therein.

Optionally, the temperature monitoring mechanism comprises a thermistor and an elongated directing element, wherein the thermistor is located at a proximal edge of the elongated directing element. The elongated directing element extends from the casing, while inserted through an opening in the arm, to allow the thermistor to abut the subject's body when the subject wears the mobile monitoring device.

According to some embodiments, the control unit further allows identifying at least one type of temperature alarming situation by comparing a currently measured body temperature of the subject with at least one predefined threshold temperature and operating a temperature alarm upon identification of the temperature alarming situation, using the alarm mechanism.

Optionally, the alarm mechanism further includes a delay mechanism for temperature alarm, which enables turning on a temperature alarm upon identification of a first temperature alarming situation, in which said measured temperature exceeds said threshold, turning the temperature alarm off after a predefined alarm period and operating the alarm again after a predefined delay period if the currently measured temperature still exceeds the threshold temperature.

Additionally or alternatively, the mobile monitoring device further comprises a transmitter, which enables transmitting monitoring related data to at least one remote unit for allowing users to view the related data and/or for outputting alarm thereby, each remote unit comprises a receiver for receiving the data and at least one output device for displaying the data and/or outputting the alarm, respectively.

According to some embodiments of the present invention, there is provided a method of monitoring physical characteristics of a subject, using a mobile monitoring device (MMD). The method comprises: attaching the MMD to a garment edge of the subject using an attaching mechanism thereof; sensing breathing characteristics of the subject by using a sensor element of the MMD, enabling to sense breathing related characteristics of the subject by converting physical vibrations into electric signals; analyzing the electric signals outputted by the sensor element for identification of at least one type of breathing alarming situation, using a control unit of the MMD to carry out such analysis; and operating an alarm device of the MMD upon identification of the respective alarming situation, wherein the control unit operates the alarm, wherein the MMD further comprises at least one arm extending from a casing of the MMD, the arm allows abutting a body portion of the subject at a first proximal end thereof and the sensor element at a second distal end thereof, the sensor element is located within the casing, and wherein the arm mechanically connects to the sensor element to allow mechanically transmitting physical movements of the arm caused by movements of the subject body to the sensor element thereby mechanically leveraging the movement.

Optionally, the identification of at least one type of breathing emergency situation comprises identification of a no-breathing situation by identifying that the amplitude of signal outputted by the sensor element is lower than a predefined threshold level consecutively over a predefined period of time.

According to some embodiments, the identification further includes rapid breathing situation identification by identifying that the frequency of breathing, which corresponds to the frequency of the output signal of the sensor element, is higher than a predefined frequency threshold over a predefined period of time.

Optionally, the method further comprises monitoring temperature of the subject using a temperature monitoring mechanism of the MMD, which comprises at least one temperature sensor. The monitoring includes receiving current temperature T0, sensed by the temperature monitoring mechanism, wherein the control unit receives the current temperature T0 from the temperature sensor; and displaying a temperature value that is associated with the receiving current temperature T0, wherein the control unit enables using at least one display device of MMD for displaying the value.

Additionally or alternatively, the method further comprises calculating a real temperature of the subject's body associated with T0, using a predefined calculation, wherein T0 is proportional to the real body temperature of the subject.

The method additionally or alternatively comprises monitoring temperature of the subject using a temperature monitoring mechanism of the MMD, which comprises at least one temperature sensor, the monitoring includes receiving current temperature T0, sensed by the temperature monitoring mechanism, wherein the control unit receives the current temperature T0 from the temperature sensor; identifying temperature related alarming situations t by checking whether the body temperature of the subject, which corresponds to T0 exceeds a predefined temperature threshold T1; and operating a temperature alarm if T0>T1.

Optionally, the method further comprises calculating a real temperature T of the subject's body associated with T0, using a predefined calculation, wherein T0 is proportional to the respective real body temperature of the subject. The identification of a temperature alarming situation includes checking whether T exceeds the predefined temperature threshold.

According to some embodiments, the analysis further includes checking whether the current body temperature T also exceeds a second predefined upper temperature threshold T2; wherein if T1<T<T2, the control unit operates a designated temperature alarm a delay alarm mechanism, according to which the alarm is frequently operated between predefined delay periods, while allowing the breathing monitoring to be operated simultaneously, and if T has reached the upper threshold T2, during the delay process or before, the control unit operates another designated temperature alarm that can only be dismantled if the user completely turns off the MMD.

According to some embodiments, the method further comprises calibrating a personal normal temperature of the specific respective subject Tₙₒᵣₘ, according to which the predefined temperature threshold is determined, by enabling a calibration process, in which the temperature of the specific subject is measured within a predefined calibration period, the measurements are used for calculating the respective personal normal temperature Tₙₒᵣₘ of the respective subject.

### BRIEF DESCRIPTION OF THE DRAWINGS

FIG. 1 shows a side view of a mobile monitoring device, according to some embodiments of the present invention.

FIG. 2 shows a side inner view of the mobile monitoring device, according to some embodiments of the present invention.

FIG. 3A shows a sensor element of the mobile monitoring device, according to some embodiments of the present invention.

FIG. 3B shows a temperature monitoring mechanism of the mobile monitoring device, according to some embodiments of the present invention.

FIG. 4 shows a front view of the mobile monitoring device, according to some embodiments of the present invention.

FIG. 5 shows a front view of the mobile monitoring device, according to some embodiments of the present invention.

FIG. 6 shows a baby subject wearing the mobile monitoring device, according to some embodiments of the present invention.

FIG. 7 is a block diagram, schematically illustrating modules of a control unit of the mobile monitoring device, according to some embodiments of the present invention.

FIG. 8 shows a mobile monitoring device having a spring mechanism, according to some embodiments of the present invention.

FIG. 9 is a flowchart, schematically illustrating a method of monitoring a human subject's breathing related physical characteristics, using a mobile monitoring device, according to some embodiments of the present invention.

FIG. 10 is a flowchart, schematically illustrating a method of monitoring a human subject's temperature related physical characteristics, using a mobile monitoring device, according to some embodiments of the present invention.

### DETAILED DESCRIPTION OF THE INVENTION

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

The present invention, in some embodiments thereof, provides a mobile monitoring device for monitoring physical characteristics of a subject especially yet not exclusively for monitoring breathing characteristics of the subject for apnea identification and optionally additional physical characteristics such as the subject's body temperature, pulse and the like. According to some embodiments of the present invention, the mobile monitoring device includes (i) a casing; (ii) an attaching mechanism such as a clip, connected to the casing, for releasably attaching the mobile monitoring device to an edge of a garment worn by the subject such as a diaper, underwear and the like; (iii) a control unit enabling to control and execute operations of the mobile monitoring device; (iv) at least one sensor element such as a piezoelectric transducer, enabling to sense breathing related characteristics of the subject such as micro-mechanical movements relating to the breathing movements of the subject, by converting physical vibrations applied thereon into electric signals; (v) an alarm mechanism for enabling to operate at least one alarm in case alarming breathing related characteristic is detected; and (vi) at least one arm extending from the casing, which includes a firs arm portion and a second arm portion connected thereto, configured to allow continuously abutting a body portion of the subject while the subject is moving once the device is attached to the subject's garment portion. The sensor element is located within the casing, where the arm mechanically connects to the sensor element to allow mechanically transmitting physical movements of the arm caused by movements of the subject's body to the sensor element thereby mechanically leveraging the movement applied upon the abutting edge of the arm.

According to some embodiments of the present invention, the mobile monitoring device is designed to be worn over the upper edge of the subject's diaper (in case of a baby subject), underwear, or trousers to allow the extending arm to touch (abut) the lower abdomen area of the subject.

A subject may be any animal being including humans, mammals and the like. The mobile monitoring device is especially yet not exclusively suitable for helping human subjects in risk of breathing irregularities such as apnea of human subjects such as babies or people having breathing problems, people who are recovering from medical procedures such as surgery and/or from a disease and the like.

According to some embodiments of the present invention, the control unit includes a digital card and/or a printed circuit enabling to receive output signals of the sensor element (e.g. output alternating current of the piezoelectric crystal) over time, analyze the pattern of movements of the body part (e.g. lower abdomen) to identify breathing irregularities. Once an irregular breathing pattern is detected, the controller operates the alarm mechanism to execute an alarm via alarm devices such as a sound alarm via audio speakers embedded in the mobile monitoring device. For example, the control unit may include a predefined algorithm for identifying breathing alarming situations, according to which only no-breathing patterns can be identified, where upon identifying no breathing (e.g. by identification of lack or low/slow of movement of the arm) the control unit turns on the alarm only if the no breathing pattern exceeds a predefined period (e.g. 20 seconds). This may be useful in case the mobile monitoring device is for baby breathing monitoring, since babies can have temporary apnea (central sleep apnea syndrome) which is normal unless the breathing is stopped or irregular for a period longer than a few seconds (typically 10-30 sec).

The sensor element (e.g. piezoelectric transducer) is located in the casing and does not directly touch the body of the subject. Instead the arm serves as a leverage for amplifying or enhancing the mechanical force applied upon the sensor element. Each movement of the body applies a force upon the abutting edge of the arm, where the arm then leverages that force enabling applying a higher force upon the sensor element, where the length of the arm determines the leveraging rate.

According to some embodiments of the present invention, the mobile monitoring device additionally includes a temperature monitoring mechanism including a directing element and a thermometer (e.g. a thermistor) configured to also abut the body of the subject once the device is worn by the subject. The temperature monitoring mechanism measures the subject's body temperature and transmits the measured values to the control unit which compares the currently measured temperature with a predefined threshold temperature (e.g. 38.0° C.) and operates (turns on) a temperature alarm once the measured temperature exceeds the threshold.

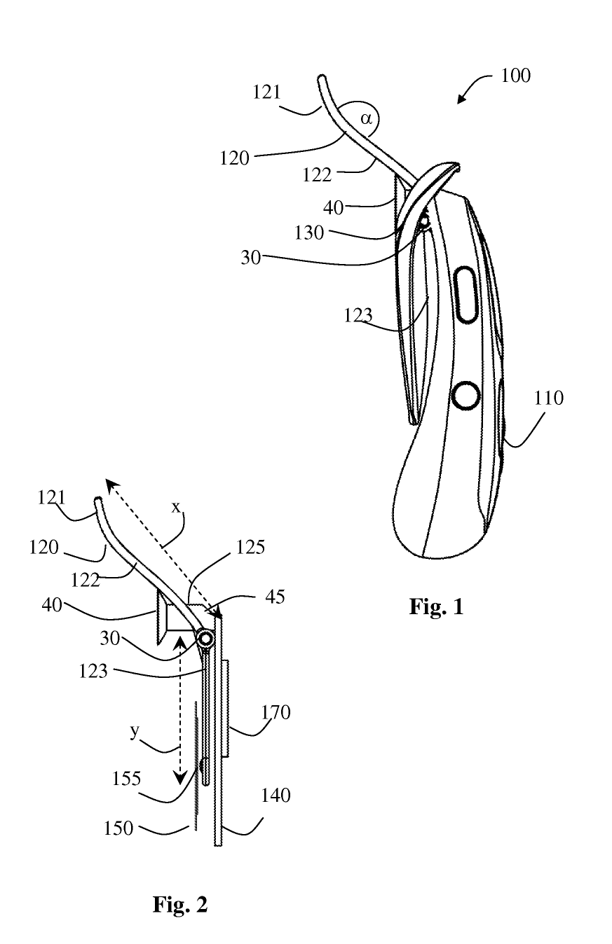

Reference is now made to FIGS. 1-2 and 4-5, schematically illustrating a mobile monitoring device (MMD) 100 for monitoring breathing related characteristics of a human subject such as a baby, according to some embodiments of the present invention. MMD 100 includes a casing 110, an arm 120 extending from casing 110, an attaching mechanism including a clip 130 configured for attaching MMD 100 to the human subject's underwear (e.g. diaper) or trousers for sensing movements of the subject's lower abdominal area, a control unit 140 including an electronic printed circuit and a sensor element 150 including a transducer 151 (e.g. a piezoelectric based transducer).

According to some embodiments, as illustrated in FIGS. 1 and 2, arm 120 includes three portions: a first arm portion 121 located externally from casing 110, a second arm portion 122 partially inside casing 110 and partially external thereto and a third arm portion 123 located inside casing 110. First arm portion 121 is integrally connected to second arm portion 122, where these two portions are angular to one another, forming an angle “α” there between, which is smaller than 180 degrees.

First arm portion 121 is designed to abut the abdomen of the human subject for allowing tactile movement detection of the abdomen. This means that first arm portion 121 is configured to touch the abdomen of the subject in a continuous manner as long as MMD 100 is worn by the subject.

First and second arm portions 121 may be made from an elastic material such as silicone, rubber, plastic or thin metal sheet and the like, to enable comfortable continuous touch over the abdominal area of the subject as well as some rotation movement span between first arm portion 121 and second arm portion 122, which also increases wearing convenience as well as better responsiveness to the subject's abdominal movements while wearing MMB 100. The elasticity level of first arm portion 121 may be determined to allow an optimal average pressure level applied by first arm portion 121 upon the subject's abdomen so that there will be a high enough pressure applied thereby for properly sensing of the subject's vibrations, while maintaining the pressure low enough for keeping it as comfortable as possible for the subject to wear over long periods (typically a few hours). All or some components of MMD 100 may be made from non-toxic materials especially for MMD 100 designed for monitoring babies.

According to some embodiments of the present invention, as illustrated in FIGS. 1 and 2, second arm portion 122 pivotally connects to third arm portion 123 via a hinge 30 enabling thereby first and second arm portions 121 and 122 to rotate about third arm portion 123 in order to balance the location of first arm portion 121 in respect to third arm portion 123. This will improve keeping a continuous touch of first arm portion 121 with the subject's body (abdomen) while the subject is moving and thereby changes the location and positioning (e.g. tilting positioning) of MMB 100 in respect to his/her body.

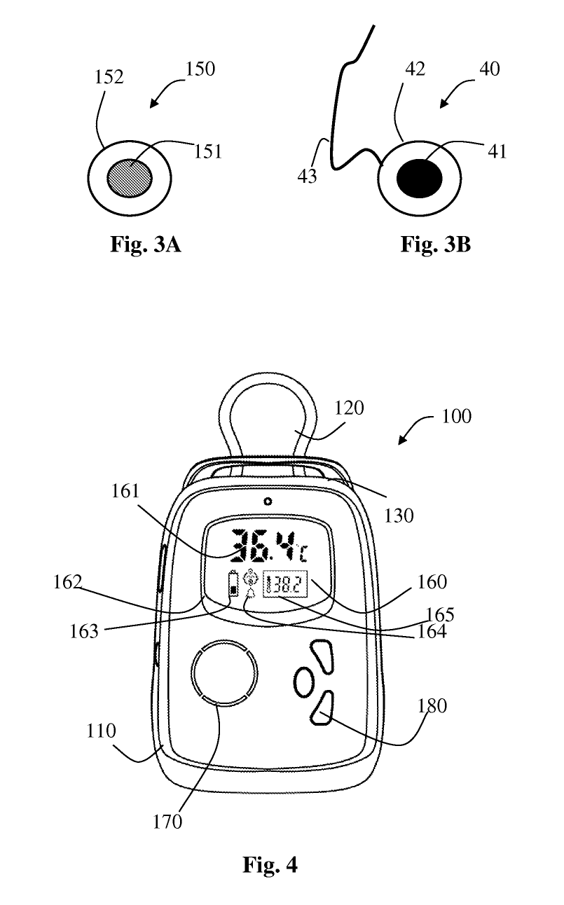

According to some embodiments of the present invention, as illustrated in FIG. 2, a distal edge of third arm portion 123 can apply force upon a designated part of sensor element 150, which includes a piezoelectric transducer 151 (see FIG. 3A) capable of converting each touch of third arm portion 123 therewith into an electronic signal proportional to the force applied thereon. To improve maintenance of the delicate crystal of piezoelectric transducer 151 a mediating element may be placed between distal edge of third arm portion 123 and transducer 151 such as a rubber stopper 155, as shown in FIG. 2. This means that each breath of the subject causing abdominal movement will cause arm portions 121-123 to move respectively. Since arm 120 leverages this movement, it may amplify the force received thereby causing the force applied by the distal edge of third arm portion 123 upon transducer 151 to be higher than that applied upon first arm portion 121 by the subject's abdomen. This technique and MMD 100 configuration improves the ultimate signal outputted by transducer 151 if compared with a system in which the transducer directly comes into contact with the subject's body (e.g. abdomen), since arm 100 leverages the received movement. The proportion between the length of third arm portion 123 “y” and the total length “x” of first and second arm portions 121 and 122, as illustrated in FIG. 2, determines the amplification rate (meaning the leveraging rate). According to some embodiments, the length of third arm 123 is linearly proportional to a multiplication of “x” meaning: y=ax, where a>1.

According to some embodiments of the present invention, as illustrated in FIG. 3, MMD 100 may further include at least one alarm output device such as a speaker 170 for outputting a sound alarm whenever an alarming situation is identified by control unit 140. For example, control unit 140 analyzes movements-pattern of arm 120 according to output pattern of transducer 151 and operates a predefined sound alarm using speaker 170, once identifying the breathing related alarming situation. For example, control unit 140 may be designed for identifying no-breathing and/or rapid breathing patterns. No breathing may be identified by identification of no output signal of transducer 151 or a low signal (e.g. low amplitude). Rapid breathing may be identified when the time between each two consecutive breaths (each two peaks of the transducer's 151 output) is smaller than a predefined threshold “t0”. Upon no-breathing/rapid breathing identification, control unit 140 operates (switches on) the sound alarm only if the pattern of no/rapid breathing stretches over more than a predefined threshold period (e.g. 20 seconds). This may be useful for baby breathing monitoring, since babies can have central sleep apnea syndrome or rapid breathing sessions, which are normal unless the breathing is stopped or is rapid for a period longer than a 10-30 sec. In this example, MMB 100 will start beeping (using speaker 170) until the caretaker of the baby voluntarily turns MMD 100 off using a designated on/off button such as button 180, for instance.

According to some embodiments, the rapid breathing identification and alarm operation further includes distinguishing between a rapid breathing, in which the breathing frequency is higher than normal yet lower than a dangerous upper level (predefined). In this case the alarm will only be operated if the rapid breathing pattern exceeds the time threshold t0. If the breathing frequency is higher than the upper threshold, the sound alarm will be immediately operated or operated after a much shorter period than t0.

Additional or alternative alarm devices and techniques may be used such as visual alarms and/or additional audio alarms. For example, in addition to the audio alarm for indicating a detected suspicious breathing situation light emitting diode (LED) indicators may be used. These LED indicators can be set to a flashing mode when a breathing alarm or any other alarm is operated such as low battery indication and the like.

According to some embodiments of the present invention, as illustrated in FIG. 3A, sensor element 150 includes piezoelectric transducer 151 and a disc 152 to which it is attached. The disc may be made from a metal such as copper, for example and may be used to amplify and/or better transmit the electric signal outputted by piezoelectric transducer 151 (e.g. by reducing the signal-to-noise ratio). The output signal may be transmitted from 152 to control unit 140 for analyzing the output pattern.

As illustrated in FIG. 2, first arm portion 121 may have a tongue-shape where it widens towards the edge thereof and narrows towards its connecting border with second arm portion 122. This may serve for allocating a higher surface area interfacing the subject's body thereby increasing sensitivity of arm 120 to the movements of the subject.

According to some embodiments of the present invention, as illustrated in FIGS. 1-2, and 3B, MMD 100 additionally includes a temperature monitoring mechanism 40, which includes, for example, a thermistor 41 where the head of thermistor 41 is surrounded by an elastic cap 42 (e.g. made of rubber or silicon based elastic materials) for allowing comfortable and flexible touch with the subject's skin for measuring temperature of the touch area/point of thermistor head 41. Thermistor 41 (typically) includes a thermometer sensor head and an electric wire enabling to connect the sensor both to a power input and to any device receiving output data/signal of the sensor. In this case, control unit 140 may connect to thermistor 40 via its wiring for receiving output of thermistor 41, which is indicative of the measured temperature of the subject at the area where thermistor 41 abuts the subject. The temperature may be measured in a continuous manner or discretely—every predefined time-interval, depending on the thermometer's abilities and definitions.

A directing element 45 as illustrated in FIGS. 1 and 2, in which thermistor 41 is inserted (as shown in FIG. 3B), covers thermistor 41 wire 43 and is inserted through a designated opening 125 (see FIG. 2) in arm 120 allowing thereby to reach the subject's abdomen for abutting thereof without disturbing the attaching of MMD 100 to the subject's garment. According to this configuration, directing element 45 extends from an upper side of MMD 100 towards the abdomen for abutting it when MMD 100 is worn.

According to some embodiments of the present invention, control unit 140 checks over time (e.g. every predefined time interval such as every few seconds or minutes) whether the measured temperature “T” exceeds a first predefined threshold “T1” for enabling indication and/or alarming the user regarding exceeding temperature. For example, if the measured temperature “T” is higher than T1=38° C., control unit 140 outputs a visual indication and/or a sound alert, which is distinguished in character from the alarm sound of the breathing alarm (e.g. a broken beep sound) and/or a visual alert indication e.g. through LED indication lamps embedded in MMD 100.

According to some embodiments, another upper threshold temperature “T2”, which is higher than T1 may be defined for alarming the user in case of reaching an endangering temperature such as T2=41/42° C. In this case, a delay mechanism may be defined, in which the temperature alarm is frequently operated upon identification of a first temperature alarming situation (e.g. when T exceeds T1 but does not yet exceeds T2: T1<T<T2). The delay mechanism includes turning the temperature alarm off after a predefined alarm period t-^{alarm} and operating the alarm again after a predefined delay period t-^{delay} if the currently measured temperature still exceeds the threshold temperature after that delay period t-^{delay} and if the user has not turned the alarm off (e.g. using a designated alarm neutralization switch). If at any point the measured temperature T exceeds the upper threshold T2 as well: T>>T1 and T>T2−control unit 140 may be set to abort the delay mechanism, operating the temperature alarm in a continuous mode requiring the user to physically turn off the entire MMD 100 for neutralizing the alarm. This may be useful in a case, for instance, in which it is required to keep the breathing monitoring operating while still monitoring the subject's temperature while there is some indication/alerting in case T1>T>T2. For example, if the baby is sleeping and his/her temperature is 38.5° C., MMD 100 indicates the alarming temperature rise by operating a sound alarm in the delay mode, for instance, that will draw the caretaker's attention. Nevertheless, to prevent the alarm from waking up the baby the alarm automatically turns off for the delay period allowing the user (caretaker of the baby for instance) to address the matter (e.g. by treating the baby) while still monitoring the baby's breathing, where the continuous sound alarm is only operated once the temperature exceeds the T2 level that can endanger the baby's life.

Alternatively, the sound alarm is only operated in a continuous mode and only when exceeding the highest temperature threshold T2 and when T1>T>T2 then only a visual indication of the temperature level is operated to avoid disturbing the subject if asleep.

According to some embodiments, MMD 100 may allow the user to select a temperature alarm mode choosing between a silent mode in which the sound alarm only operates upon exceeding the highest temperature threshold T2 and a normal mode, which includes the delay mechanism as discussed above.

Additional or alternative sensors and sensor types may be used for sensing characteristics relating to temperature of the body of the subject such as infra-red (IR) based thermometers, mercury based thermometers and the like.

MMD 100 may be used for monitoring additional or alternative physical characteristics of the subject such as body humidity level, pulse or any other biological characteristic of the subject indicative of the subject's physical condition. For each characteristic, different one or more sensor elements may be required. Each such sensor may be embedded in MMD 100 as an integral part thereof allowing indication of the measured characteristics or parameters associated therewith, identification of alarming situations relating to each such characteristic and/or alarm mechanisms that allow alarming the user (E.g. the caretaker) regarding such identified alarming situations.

FIG. 4 schematically illustrates MMD 100 having an LCD display unit 160 embedded therein. Display unit 160 connects to control unit 140 for receiving display related data there from and allows displaying monitoring related parameters such as measured or evaluated body temperature T of the subject 161, monitored breathing indication 165 indicative that the breathing is monitored, an alarm indication 163 relating to either the breathing alarm(s), the temperature alarm(s) or all. Additionally, display unit 160 displays battery status 162. Display unit 160 may enable displaying of other device related features such as the threshold temperature value of T1 165 and/or T2.

According to some embodiments of the present invention, MMD 100 further allows the user to set threshold parameters such as the lower threshold temperature T1. This may be enabled by adding input and/or control buttons to MMD 100 such as buttons 180 or by using a touch screen as display unit 160 allowing inputting or setting MMD 100 features through a predefined designated menu. A minimum value of T1 may be predefined “T1min: where the user may be prevented from setting T1 to a value that is lower than T1min, to avoid false alarms. The minimum value T1min may be defined as the highest normal temperature value (e.g. 37.5° C. for human subjects).

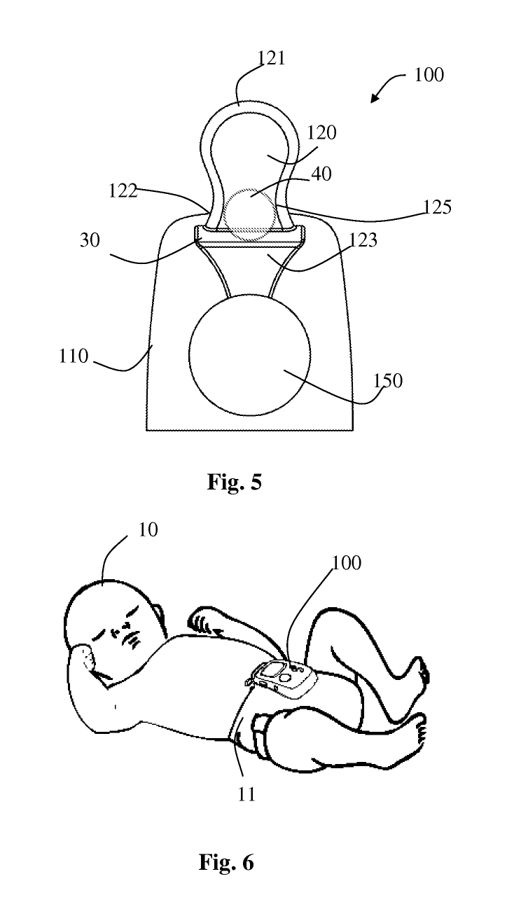

FIG. 5 schematically shows a front view of MMD 100 showing how third arm portion 123 is connected to sensor element 150 and how the edge of temperature measuring mechanism 40 emerges from opening 125 located in second arm portion 122.

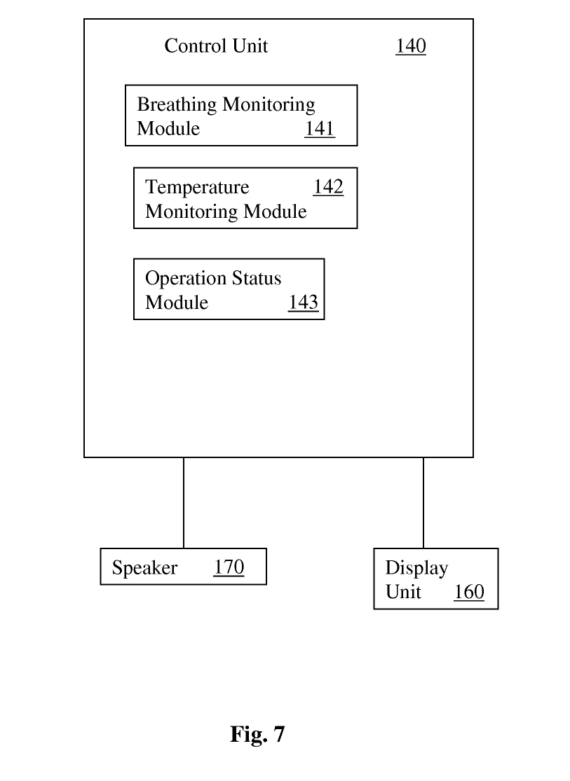

FIG. 6 schematically shows how MMD 100 attaches to an upper edge of a diaper 11 worn by a baby subject 10 enabling thereby arm 120 and temperature measuring mechanism 40 to abut baby's 10 lower abdominal area for monitoring breathing and temperature of baby 10.

Reference is now made to FIG. 7, which is a block diagram schematically illustrating modules of control unit 140 and its connection to alarm and display components, according to some embodiments of the present invention. Control unit 140 includes a breathing monitoring module 141, a temperature monitoring module 142 and an operation status module 143.

According to some embodiments, breathing monitoring module 141 receives output signals of sensor element 150, such as electric signals outputted by piezoelectric transducer 150, indicative of signal amplitude over time pattern and analyzes this pattern to identify breathing alarming situations. Upon a breathing alarming situation, breathing monitoring module 141 operates a sound alarm by sending speaker 160 a signal indicative of a predefined sound alarm output pattern associated with the specific breathing alarming situation. For example, in case of a no-breathing situation the sound alarm may be a continuous beep sound of a predefined first frequency and in case of a rapid breathing alarming situation the sound alarm may be a continuous beep sound of a second frequency. Alternatively, the sound alarm for a breathing alarming situation may be the same for both alarming cases where the type of the breathing situation (no-breathing/rapid) may be indicated upon a display unit 170 embedded in MMD 100 (as will be described below).

Breathing monitoring module 141 can include hardware components and optionally additional software components to enable it functionality as described above.

According to some embodiments of the present invention, temperature monitoring module 142 receives output temperature measurements from the output of thermistor 41, optionally analyzes this output and enables displaying the measured temperature T0 over display unit 170, evaluating the real body temperature T of the subject from the measured temperature T0, analyzing the temperature T for identification of temperature alarming situations (as explained above) and operating alarm options relating to such situations such as operating a sound alarm using speaker 170 and displaying the body temperature T and optionally temperature alarm relating indications over display unit 160, which electronically connects to control unit 140.

According to some embodiments, the sound of the temperature alarm(s) may be different from that of the breathing alarm(s) to allow the user to distinguish each alarm type. For example, the temperature sound alarm may be broken while the breathing alarm continuous.

Temperature monitoring module 141 can include hardware components and optionally additional software components to enable it functionality as described above.

According to some embodiments of the present invention, operation status module 143 is configured for identifying the power status of MMD 100 and indicating it over display unit 160 and/or over a separate indicator such as an indication lamp (e.g. LLD lamp) that is turned on whenever MMD power is on. Operation status module 143 additionally identifies and indicates battery status of MMD 100 for allowing indicating the user when the battery is low and should soon be replaced or charged.

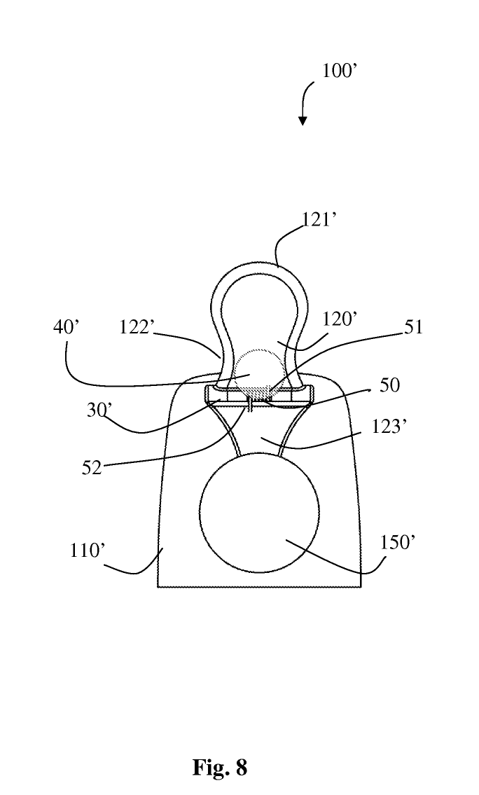

Reference is now made to FIG. 8, schematically illustrating another MMD 100′ that includes a spring 50, according to other embodiments of the present invention. All components of MMD 100′ such as arm 120′ and its portions 121′,122′ and 123′, hinge 30′, casing 110′, temperature monitoring mechanism 40′ and sensor element 150′ are equivalent or similar to components 120, 121,122, hinge 30 and 123, 110, 40 and 150 of MMD 100 as described above. MMD 100′ includes spring 50 located in the location of hinge 30′, wherein hinge 30′ is inserted through spring 50. A first end 51 of spring 50 connects to a lower end of second arm portion 122′ and a second end 52 of spring 50 connects to the upper end of third arm portion 123′. Spring 50 may be a coiled spring s illustrated in FIG. 8 or any other spring type as long as it is connected to MMD 100′ in a manner that allows increasing its compression by pivotally pulling first and second arm portions 121′ and 122′ towards third arm portion 123′.

Spring 50 may enable improving controlling the pressure applied by arm 120′ upon the subject's body for a more comfortable wearing of MMD 100′ and yet efficient monitoring thereby.

According to some embodiments of the present invention, spring 50 may allow using more stiff/non flexible substances for arm portions such as 121 or the entire arm 120, since the flexibility added or enabled by spring 50 may be enough for improving the mechanical responsiveness of MMD 100 to the body movements of the subject. In this case even a stiff material of arm 120 would not disturb the subject if a very flexible spring is chosen.

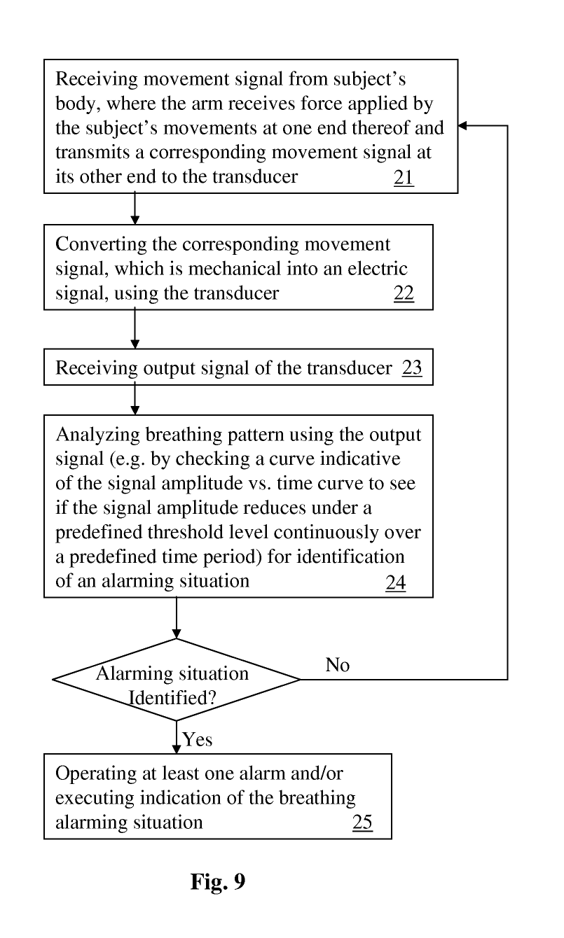

Reference is now made to FIG. 9, which is a flowchart, schematically illustrating a process of monitoring breathing of the subject using a MMD such as MMD 100/100′ attached to a subject's underwear garment edge, according to some embodiments of the present invention.

MMD receives movement signal from subject's body, where the arm of the MMD receives force applied by the subject's movements at a proximal end thereof (proximal in the sense that it is closer and abutting the subject's body) and transmits a corresponding movement signal to the transducer at a distal end thereof 21. The force applied upon the arm is leveraged thereby so that the arm applies a greater force at its distal end upon the transducer's surface. The transducer, in turn, converts the mechanical force applied thereupon by the arm into an electric signal 22 that corresponds in amplitude to the applied force 22. The control unit receives this electric signal and analyzes it over time to identify breathing alarming situation(s) according to predefined identification and analysis methods 23 such as methods for identifying no-breathing situations and/or rapid breathing situations as described above. If an alarming breathing situation is identified 24, the control unit operates an alarm and/or executes indication of the alarming situation using devices such as audio and/or visual output device(s) of MMD to output the alarm and/or the indication 25.

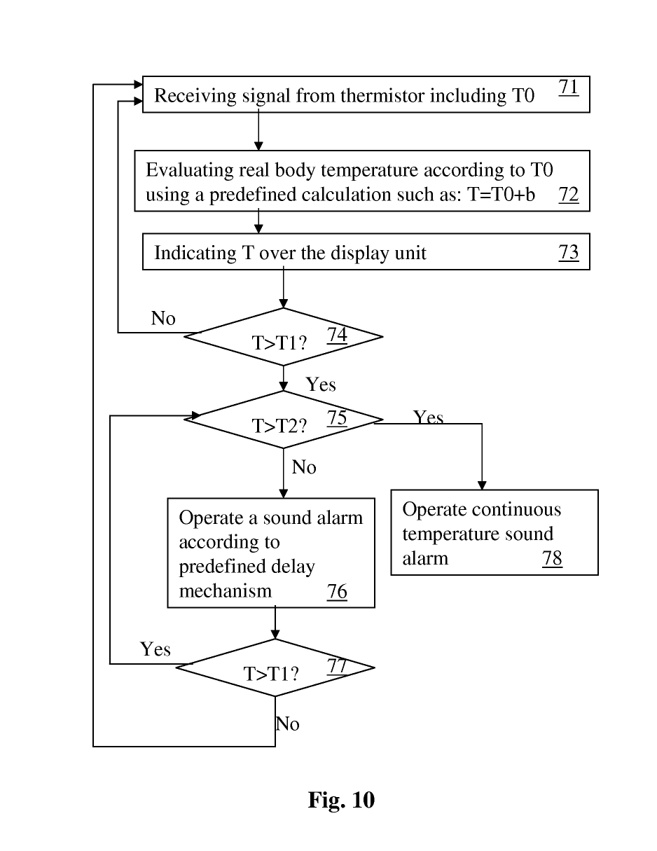

Reference is now made to FIG. 10, which is a flowchart, schematically illustrating a process of monitoring body temperature of the subject, according to some embodiments of the present invention.

Current temperature measurement T0, sensed by the temperature monitoring mechanism is received by the control unit 71, which calculates a real temperature of the subject's body associated therewith, according to a predefined calculation 72. The calculation is done in cases in which MMD enables abutting an area of the subject's body whose temperature does not properly reflect the average body temperature. This temperature T0 is, however, proportional to the body temperature and a conversion is required to evaluate the real current body temperature T of the subject. This conversion is based on statistical study of the relation between the temperatures measured in the respective body area (e.g. lower abdominal area) and the temperature measured in more reliable areas such as the subject's mouth, rectum or ear. For example, the calculation is done by using a conversion equation wherein: T=T0+Δ, where B is a constant predefined parameter or T=AT0+Δ, wherein A is a predefined coefficient. The evaluated current body temperature T may be indicated over the display unit of MMD 73.

The control unit than checks whether the evaluated current body temperature T exceeds a first lower temperature threshold T1 74, where T1 can be a temperature level that exceeds normal values in a predefined delta value (e.g. if the normal value is 37.5° C. then T1 may be set to a value of 38.2° C.). If T>T1, the control unit checks whether it also exceeds an upper temperature threshold T2 75, which is predefined and reflects a temperature that can endanger the subject or slightly under such value (e.g. T2=41.5° C. where 42° C. is a temperature typically requiring urgent medical treatment and observation for human subjects).

If T1 is exceeded but T2 is not meaning: T1<T<T2, the control unit operates a designated temperature alarm and/or indication including the delay mechanism as described above 76, while allowing to still check if the temperature has reached the upper threshold T2 77 during the delay mechanism process. If the temperature has exceeded T2 during the delay process or before 78, the control unit operates another designated temperature alarm that can only be dismantled if the user completely turns off the MMD using a designated on/off button or switch.

According to some embodiments of the present invention, temperature monitoring module 142 enables studying the personal temperature behavior of the subject in relation to the standard normal body temperature by, for example, enabling and requiring a predefined personal calibration procedure, in which the differences between the measured temperature of the specific subject T0 and the predefined standard normal temperature (e.g. Tₙₒᵣₘ=36.7° C.) are measured and saved over time to calculate an optimal difference level Δ that will be considered in the calculation of the real temperature T=T0+Δ as mentioned above.

To do so, the user may be required to operate MMD 100 for an initial predefined temperature calibration period when the subject is of normal temperature. For example, in case the subject is a baby, the caretaker may be required to attach MMD 100 to the baby's diaper edge for an initial period of a few minutes, only when the baby is of normal temperature. MMD 100 automatically calibrates itself by measuring the abdominal area temperature of the baby T0 every few minutes, saving each measured value and then either: (i) calculating an average T0, which may then be defined as the normal temperature to which MMD 100 compares the currently measured T0 with to evaluate exceeding values; or (ii) assuming that the baby is of a normal standard temperature of Tnorm and calculating an average difference Δ between the predefined Tnorm and the measured abdominal temperature to define thereby the Δ parameter for adding to each currently measured T0 for calculation of the real temperature T, as specified above.

According to some embodiments of the present invention the MMD may enable transmitting monitored related data to one or more remote units that may be able to display monitored related data such as body temperature, breathing monitoring indication and the like and/or operate alarm using alarm devices such as a speaker or LED indicators embedded in the remote unit(s). The communication between the MMD and each remote unit may be a wireless communication (e.g. using one or more designated RF wireless communication channels). This will allow a caretaker user to hear the alarm or see the monitored parameters when being located at a substantial distance from the MMD (and the subject wearing it). The communication range may be determined according to statistical information (e.g. of average highest distance between rooms in households/apartments and the like) and according to safety standards.

To allow the wireless communication, the MMD may further include a transmitter such as an RF transmitter, where each remote unit may include a corresponding receiver.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments and/or by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

Although the invention has been described in detail, nevertheless changes and modifications, which do not depart from the teachings of the present invention, will be evident to those skilled in the art. Such changes and modifications are deemed to come within the purview of the present invention and the appended claims.

1. A mobile monitoring device for monitoring physical characteristics of a subject, said mobile monitoring device comprises:
i) a casing;
ii) an attaching mechanism, connected to said casing, for releasably attaching said mobile monitoring device to a garment worn by the subject;
iii) a control unit enabling to control and execute operations of said mobile monitoring device and identifying breathing related alarming situations;
iv) at least one sensor element enabling to sense breathing related characteristics of the subject by converting physical vibrations into electric signals, said sensor element is operatively connected to said control unit;
v) an alarm output device for enabling to operate at least one alarm in case a breathing related alarming situation is identified; and
vi) at least one arm extending from said casing, said arm allows abutting a body portion of the subject at a first proximal end thereof and said sensor element at a second distal end thereof,
wherein said sensor element is located within said casing, said arm mechanically connects to said sensor element to allow mechanically transmitting physical movements of said arm caused by movements of the subject body to said sensor element thereby mechanically leveraging said movement.

2. The mobile monitoring device according to claim 1, wherein said arm comprises a first arm portion and a second arm portion connected thereto, said first arm portion is made from a flexible material, wherein said first arm portion is position angularly to said second arm portion to allow comfortable abutting of the subject's body while wearing said mobile monitoring device.

3. The mobile monitoring device according to claim 2, wherein said arm further comprises a third arm portion pivotally connected to said second arm portion.

4. The mobile monitoring device according to claim 3, wherein said third arm portion pivotally connects to said second arm portion via a hinge or is integrally connected thereto and is made from a flexible material allowing movement of said third arm portion in relation to said second arm portion thereby.

5. The mobile monitoring device according to claim 3, wherein said third arm portion is longer than the overall length of said first and second arm portions combined.

6. The mobile monitoring device according to claim 3, wherein a coiled spring connects to said third arm portion at one end and to said second arm portion at another end.

7. The mobile monitoring device according to claim 1, wherein said sensor element comprises a piezoelectric transducer enabling to convert mechanical force applied upon a surface thereof by said second distal end of said arm into an electric signal.

8. The mobile monitoring device according to claim 1, wherein said attaching mechanism comprises a clip for releasably attaching said mobile monitoring device to said garment.

9. The mobile monitoring device according to claim 1 further comprising at least one speaker for outputting sound alarms, said control unit allows identification of a breathing related alarming situation by analyzing movement pattern of said arm and operating the sound alarm once identifying the breathing related alarming situation.

10. The mobile monitoring device according to claim 1 further comprising a temperature monitoring mechanism, said temperature monitoring mechanism comprises at least one temperature sensor operatively connected to said control unit enabling measuring temperature of an area of the subject's body, wherein said mobile monitoring device enables presenting measured temperature over a designated display unit embedded therein.

11. The mobile monitoring device according to claim 10, wherein said temperature monitoring mechanism comprises a thermistor and an elongated directing element, wherein said thermistor is located at a proximal edge of said elongated directing element, said elongated directing element extends from said casing, while inserted through an opening in said arm, to allow said thermistor to abut the subject's body when the subject wears said mobile monitoring device.

12. The mobile monitoring device according to claim 10, wherein said control unit further allows identifying at least one type of temperature alarming situation by comparing a currently measured body temperature of the subject with at least one predefined threshold temperature and operating a temperature alarm upon identification of said temperature alarming situation, using said alarm mechanism.

13. The mobile monitoring device according to claim 12, wherein said alarm mechanism further includes a delay mechanism for temperature alarm, which enables turning on a temperature alarm upon identification of a first temperature alarming situation, in which said measured temperature exceeds said threshold, turning the temperature alarm off after a predefined alarm period and operating the alarm again after a predefined delay period if the currently measured temperature still exceeds said threshold temperature.

14. The mobile monitoring device according to claim 1 further comprising a transmitter and enables transmitting monitoring related data to at least one remote unit for allowing users to view said related data and/or for outputting alarm thereby, each said remote unit comprises a receiver for receiving said data and at least one output device for displaying said data and/or outputting said alarm, respectively.

15. A method of monitoring physical characteristics of a subject, using a mobile monitoring device (MMD),
said method comprises:
(i) attaching said MMD to a garment edge of said subject using an attaching mechanism thereof;
(ii) sensing breathing characteristics of the subject by using a sensor element of said MMD, enabling to sense breathing related characteristics of the subject by converting physical vibrations into electric signals;
(iii) analyzing the electric signals outputted by said sensor element for identification of at least one type of breathing alarming situation, using a control unit of said MMD to carry out such analysis; and
(iv) operating an alarm device of said MMD upon identification of said respective alarming situation, wherein said control unit operates said alarm,
wherein said MMD further comprises at least one arm extending from a casing of said MMD, said arm allows abutting a body portion of the subject at a first proximal end thereof and said sensor element at a second distal end thereof, said sensor element is located within said casing, and
wherein said arm mechanically connects to said sensor element to allow mechanically transmitting physical movements of said arm caused by movements of the subject body to said sensor element thereby mechanically leveraging said movement.

16. The method according to claim 15, wherein said identification of at least one type of breathing emergency situation comprises identification of a no-breathing situation by identifying that the amplitude of signal outputted by said sensor element is lower than a predefined threshold level consecutively over a predefined period of time.

17. The method according to claim 16, wherein said identification further includes rapid breathing situation identification by identifying that the frequency of breathing, which corresponds to the frequency of the output signal of said sensor element, is higher than a predefined frequency threshold over a predefined period of time.

18. The method according to claim 15 further comprising monitoring temperature of said subject using a temperature monitoring mechanism of said MMD, which comprises at least one temperature sensor, said monitoring includes (i) receiving current temperature T0, sensed by said temperature monitoring mechanism, wherein said control unit receives said current temperature T0 from said temperature sensor; and
(ii) displaying a temperature value that is associated with the receiving current temperature T0, wherein said control unit enables using at least one display device of MMD for displaying said value.

19. The method according to claim 18 further comprising calculating a real temperature of the subject's body associated with T0, using a predefined calculation, wherein T0 is proportional to said respective real body temperature of the subject.

20. The method according to claim 15 further comprising monitoring temperature of said subject using a temperature monitoring mechanism of said MMD, which comprises at least one temperature sensor, said monitoring includes
(i) receiving current temperature T0, sensed by said temperature monitoring mechanism, wherein said control unit receives said current temperature T0 from said temperature sensor;
(ii) identifying temperature related alarming situations t by checking whether the body temperature of the subject, which corresponds to T0 exceeds a predefined temperature threshold T1; and
(iii) operating a temperature alarm if T0>T1.

21. The method according to claim 20 further comprising calculating a real temperature T of the subject's body associated with T0, using a predefined calculation, wherein T0 is proportional to said respective real body temperature of the subject, said identification of a temperature alarming situation includes checking whether T exceeds said predefined temperature threshold.

22. The method according to claim 21, wherein said analysis further includes checking whether the current body temperature T also exceeds a second predefined upper temperature threshold T2; wherein if T1<T<T2, the control unit operates a designated temperature alarm a delay alarm mechanism, according to which the alarm is frequently operated between predefined delay periods, while allowing the breathing monitoring to be operated simultaneously, and if T has reached the upper threshold T2, during the delay process or before, the control unit operates another designated temperature alarm that can only be dismantled if the user completely turns off the MMD.

23. The method according to claim 20 further comprising calibrating a personal normal temperature of the specific respective said subject Tₙₒᵣₘ, according to which said predefined temperature threshold is determined, by enabling a calibration process, in which the temperature of the subject is measured within a predefined calibration period, said measurements are used for calculating said respective personal normal temperature Tₙₒᵣₘ of the respective subject.
